(19) United States
(12) Patent Application Publication
(10) Pub. No.: US 2023/0137524 A1
(43) Pub. Date: May 4, 2023

(54) COSMETIC PRODUCT COMPRISING OIL DISCONTINUOUS PHASE

(71) Applicant: The Procter & Gamble Company, Cincinnati, OH (US)

(72) Inventors: Naohisa Yoshimi, Singapore (SG); Michiko Ohya, Singapore (SG)

(21) Appl. No.: 17/979,312

(22) Filed: Nov. 2, 2022

Related U.S. Application Data
(60) Provisional application No. 63/290,626, filed on Dec. 16, 2021.
(60) Provisional application No. 63/275,416, filed on Nov. 3, 2021.

(51) Int. Cl. A61K 8/06

(52) U.S. Cl. CPC A61K 8/062; A61K 2800/48; A61K 2800/882; A61K 2800/10

(57) ABSTRACT

A cosmetic composition that having an aqueous continuous phase and an oil discontinuous phase dispersed in the aqueous continuous phase. The oil discontinuous phase can have a particle size of from about 0.01 mm to about 20 mm. The oil discontinuous phase can be dispersed for at least 24 hours and the composition can be free of polymeric encapsulation. The cosmetic composition can provide prolonged dispersion of the dispersed oil phases, especially prolonged dispersion of the visible dispersed oil phases without the use of polymeric encapsulation and/or oil-absorbing porous particulates for dispersions.

### FIELD OF THE INVENTION

The present invention is directed to a cosmetic composition comprising an aqueous continuous phase and an oil discontinuous phase dispersed in the aqueous continuous phase, wherein the oil discontinuous phase has a particle size of from about 0.01 mm to about 20 mm, wherein the oil discontinuous phase is dispersed for at least 24 hours, and wherein the composition is free of polymeric encapsulation. The cosmetic composition of the present invention provides prolonged dispersion of the dispersed oil phases, especially providing prolonged dispersion of the visible dispersed oil phases, especially without the use of polymeric encapsulation and/or oil-absorbing porous particulates for dispersions.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damages in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin i.e., flaking, scaling, dryness, and roughness.

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling, spots, and other histological changes typically associated with the aging of skin or environmental damage to human skin. Consumers prefer topically applied products since they are not only effective, but also safe and pleasant to use.

For example, there are some dual phase products which contain both oil and water phases in one bottle wherein the oil phase usually floating on the surface of a water phase, and which are recommended for consumers to shake the bottle before use to make the oil phase dispersed in the water phase. Some of such oil dispersions are visible. However, such oil phase dispersions can be observed usually for only a few hours. There exists a need for such dual phase products which can sustain the dispersed oil phase for a longer period of time.

On the other hand, in view of sustained dispersion and/or visible dispersion of oil phases in water phases, the uses of polymer encapsulations and/or oil-absorbing porous particulates are known. However, in order to contain a certain amount of the oils in the composition, relatively higher amount of such polymer encapsulations and/or oil-absorbing porous particulates need to be used. There also exists a need for oil dispersion to be sustained for a longer period of time and/or to be visible, without the uses of polymer encapsulations and/or oil-absorbing porous particulates.

Based on the foregoing, there is a need for a cosmetic product providing prolonged dispersion of the dispersed oil phases, especially providing prolonged dispersion of the visible dispersed oil phases, especially without the use of polymeric encapsulation and/or oil-absorbing porous particulates for dispersions.

### SUMMARY OF THE INVENTION

The present invention is directed to a cosmetic composition comprising an aqueous continuous phase and an oil discontinuous phase dispersed in the aqueous continuous phase, wherein the oil discontinuous phase has a particle size of from about 0.01 mm to about 20 mm, wherein the oil discontinuous phase is dispersed for at least 24 hours, and wherein the composition is free of polymeric encapsulation.

The cosmetic composition of the present invention provides prolonged dispersion of the dispersed oil phases, especially providing prolonged dispersion of the visible dispersed oil phases, especially without the use of polymeric encapsulation and/or oil-absorbing porous particulates for dispersions.

### DETAILED DESCRIPTION OF THE INVENTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25 ° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions described herein can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread a composition onto a human skin surface such as the epidermis.

“Derivative,” herein, means amide, ether, ester, amino, carboxyl, acetyl, and/or alcohol derivatives of a given compound.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Cosmetic composition” means a composition comprising a cosmetic agent. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Synergy,” and variations thereof, means that the effect provided by a combination of two or more materials is more than the additive effect expected for these materials.

“Treatment period,” as used herein, means the length of time and/or frequency that a material or composition is applied to a target skin surface.

### Cosmetic Composition

The cosmetic composition can comprise an aqueous continuous phase and an oil discontinuous phase dispersed in the aqueous continuous phase, wherein the oil discontinuous phase has a particle size of from about 0.1 mm to about 20 mm, wherein the oil discontinuous phase is dispersed for at least 24 hours, and wherein the composition is free of polymeric encapsulation.

### Free of Polymeric Encapsulation

The composition can be free of polymeric encapsulation. Polymeric encapsulations herein mean those having polymeric outer shells which encapsulate inner materials. Even without polymeric encapsulation, the oil discontinuous phase and the aqueous continuous phase can make the oil discontinuous phase dispersed for longer time in the aqueous continuous phase.

### Aqueous Continuous Phase

Aqueous continuous phase can be included in the composition at a level by weight of the composition of from about 10% to about 99.99%, alternatively from about 30% to about 99.95%, alternatively from 50% about to about 99.9%, alternatively from about 60% to about 99.9%, alternatively from about 70% to about 99.9%.

### Ingredients in Aqueous Continuous Phase

The Aqueous continuous phase contains an aqueous carrier and preferably hydrophilic thickeners.

### Aqueous Carrier/Water

The composition, and the aqueous continuous phase comprises an aqueous carrier. The aqueous carrier can be substantially water. Deionized water can be used. Water from natural sources including mineral cations can also be used, depending on the desired characteristic of the composition.

The water can be in included in the aqueous continuous phase at a level of from about 30% to about 99.95%, alternatively from about 50% to about 99.9%, alternatively from about 60% to about 99.9%, by weight of the aqueous continuous phase.

The water can be included in the composition at a level of from about from about 10% to about 99.9%, alternatively from about 30% to about 99.5%, alternatively from 50% about to about 99.5%, alternatively from about 60% to about 99.5%, alternatively from about 70% to about 99.5%, by weight of the composition.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils).

### Hydrophilic Thickeners

The aqueous continuous phase can contain a hydrophilic thickener.

Hydrophilic thickeners can be included in the aqueous phase, at a level by weight of the aqueous phase, alternatively from about 0.01% to about 5%, alternatively from about 0.03% to about 4%, and alternatively from about 0.05% to about 3%.

Any hydrophilic thickeners can be used herein as long as it is one that is normally used in cosmetic products. The aqueous continuous phase containing the hydrophilic thickener can meet the conditions described below under the title “PHYSICAL PARAMETERS OF THE PHASES”. Hydrophilic thickeners useful herein can be: those with associating ability to oils; those without associating ability to oils; and mixtures thereof.

Hydrophilic thickeners without associating ability to oil include, for example: Carbomer (for example, those having tradenames of Carbopol Ultrez 10, Carbopol Ultrez 30 available from Lubrizol); Acrylates/C10-30 Alkyl Acrylate Crosspolymer (for example, those having tradenames of Carbopol Ultrez 20, Carbopol Ultrez 21, Carbopol ETD2020 available from Lubrizol); Xanthan Gum (for example, Keltrol CG available from CP Keloco); Hydroxyethylcellulose (for example, that having a tradename of Natrosol250GR available from Ashland); Sodium Polyacrylate (for example, that having a tradename of Cosmedia SP available from BASF); Sodium Polyacrylate Starch (for example, those having tradenames of Makimmouse-12, Makimmouse-25 available from Daito Kasei); Caesalpinia Spinosa Gum; Polyvinylpyrrolidone; Carageenan (for example, those having tradenames of Genugel CG130, Genugel CG131 available from CP Kelco); Pecthin (for example, those having tradenames of Genuphresh RB, Genufresh DF available from CP Kelco); Gellan Gum (for example, that having a tradename of Kelcogel CG-HA available from CP Kelco); Ammonium Acryloyldimethyltaurate/VP Copolymer (for example, that having a tradename of Aristoflex AVC available from Clariant); Sodium Acryloyldimethyltaurate/VP Copolymer (for example, that having a tradename of Aristoflex AVS available from Clariant).

Among them, preferred are: Carbomer (for example, those having tradenames of Carbopol Ultrez 10, Carbopol Ultrez 30 available from Lubrizol); Acrylates/C10-30 Alkyl Acrylate Crosspolymer (for example, those having tradenames of Carbopol Ultrez 20, Carbopol Ultrez 21, Carbopol ETD2020 available from Lubrizol); Xanthan Gum (for example, Keltrol CG available from CP Keloco); Hydroxyethylcellulose (for example, that having a tradename of Natrosol250GR available from Ashland); Sodium Polyacrylate Starch (for example, those having tradenames of Makimmouse-12, Makimmouse-25 available from Daito Kasei); Carageenan (for example, those having tradenames of Genugel CG130, Genugel CG131 available from CP Kelco); Ammonium Acryloyldimethyltaurate/VP Copolymer (for example, that having a tradename of Aristoflex AVC available from Clariant); Sodium Acryloyldimethyltaurate/VP Copolymer (for example, that having a tradename of Aristoflex AVS available from Clariant).

Among them, more preferred are: Carbomer (for example, those having tradenames of Carbopol Ultrez 10, Carbopol Ultrez 30 available from Lubrizol); Acrylates/C10-30 Alkyl Acrylate Crosspolymer (for example, those having tradenames of Carbopol Ultrez 20, Carbopol Ultrez 21, Carbopol ETD2020 available from Lubrizol); Xanthan Gum (for example, Keltrol CG available from CP Keloco); Carageenan (for example, those having tradenames of Genugel CG130, Genugel CG131 available from CP Kelco); Ammonium Acryloyldimethyltaurate/VP Copolymer (for example, that having a tradename of Aristoflex AVC available from Clariant).

Hydrophilic thickening polymer with associating ability to oils include, for example: Hydrophobically modified acrylates/C10-30 Alkyl Acrylate Crosspolymer (for example, those having tradenames of Pemulen TR-1, Pemulen TR-2, Pemulene EZ-4U, Carbopol 1382 available from Lubrizol); Polyacrylate Crosspolymer-6 (for example, that having a tradename of Sepimax Zen available from Seppic); Polyacrylate Crosspolymer-11 (for example, that having a tradename of Aristoflex Velvet available from Clariant); Acrylates/Vinyl Isodecanoate Crosspolymer (for example, that having a tradename of Stabylen30 available from 3VSigma); Ammonium Acryloyldimethyltaurate/Beheneth-25 Methacrylate Crosspolymer (for example, that having a tradename of Aristoflex HMB available from Clariant); Hydrophobically-modified Hydroxyethylcellulose (for example, that having a tradename of Natrosol Plus 330 available from Ashland); Polyacrylate-1 Crosspolymer (for example, that having a tradename of Carbopol Aqua CC available from Lubrizol).

Among them, preferred are: Hydrophobically modified acrylates/C10-30 Alkyl Acrylate Crosspolymer (for example, those having tradenames of Pemulen TR-1, Pemulen TR-2, Pemulene EZ-4U, Carbopol 1382 available from Lubrizol); Polyacrylate Crosspolymer-6 (for example, that having a tradename of Sepimax Zen available from Seppic); Polyacrylate Crosspolymer-11 (for example, that having a tradename of Aristoflex Velvet available from Clariant); Acrylates/Vinyl Isodecanoate Crosspolymer (for example, that having a tradename of Stabylen30 available from 3VSigma); Ammonium Acryloyldimethyltaurate/Beheneth-25 Methacrylate Crosspolymer (for example, that having a tradename of. Aristoflex HMB available from Clariant).

Among them, more preferred are: Hydrophobically modified acrylates/C10-30 Alkyl Acrylate Crosspolymer (for example, those having tradenames of Pemulen TR-1, Pemulen TR-2, Pemulene EZ-4U, Carbopol 1382 available from Lubrizol); Polyacrylate Crosspolymer-6 (for example, that having a tradename of Sepimax Zen available from Seppic); Polyacrylate Crosspolymer-11 (for example, that having a tradename of Aristoflex Velvet available from Clariant); Acrylates/Vinyl Isodecanoate Crosspolymer (for example, that having a tradename of Stabylen30 available from 3VSigma).

### Other Ingredients in Aqueous Phase

The aqueous continuous phase may include one or more other ingredients known for use in topical cosmetic compositions, provided that such other ingredients do not unacceptably alter the desired benefits and/or properties of the aqueous continuous phase. Such other ingredients include, for example, those described under the title of “OTHER INGREDIENTS”.

Any ingredients can be incorporated regardless of hydrophobicity/hydrophilicity, as long as such ingredients do not prevent/disturb formation and dispersion of oil discontinuous phase. Any water-soluble, partially water-soluble ingredients (such as those having a water-solubility of about 0.5 g/L or more at 25° C.), water-insoluble chemicals can be added.

Representative other ingredients in the aqueous phase include, for example:
Polyols such as glycerin, butylene glycol, propylene glycol, pentandiol, hexandiol, xylitol, trehalose, inositol; Preservatives such as parabens, DMDM hydantoin; Electrolyte such as sodium chloride, potassium chloride, calcium chloride;
pH modifier such as citric acid, sodium citrate, lactic acid, sodium lactate, glucono delta lactone;
Chelating agent such as disodium EDTA;
Skin conditioning agent such as ascorbic acid, DL-panthenol, Niacinamide;
Emulsifiers can be incorporated such as polysorbate 20, polysorbate 40, polysorbate 60, Laureth-4, Laureth-7, PEG-100 stearate, glyceryl monostearate, PEG-11 methyl ether dimethicone, PEG-10 dimethicone.

### Oil Discontinuous Phase

The oil discontinuous phase can be included in the composition at a level by weight of the composition of from about 0.01% to about 90%, alternatively from about 0.05% to about 70%, alternatively from 0.1% about to about 50%, alternatively from about 0.1% to about 40%, alternatively from about 0.1% to about 30%.

The oil discontinuous phase is dispersed in the aqueous phase, and the oil discontinuous phase has a particle size of from about 0.01 mm to about 20 mm, alternatively 0.05 mm to about 10 mm, alternatively from about 0.1 mm to 5 mm. The oil discontinuous phase is dispersed for at least 24 hours, at least 3 days, at least for 5 days, alternatively for at least 1 week. The particle size used herein means a volume-based average particle size.

### Ingredients in Oil Discontinuous Phase

### Oily Components

Oily components are included in the oil phases at a level of from about 20% to about 100%, alternatively from about 30% to about 100%, alternatively from about 40% to about 100%, alternatively from about 50% to about 100%, by weight of the oil phases.

The oily component can be in included in the composition at a level of from about 0.01% to about 90%, alternatively from about 0.05% to about 70%, alternatively from about 0.1% to about 50%, alternatively from about 0.1% to about 40%, alternatively from about 0.1% to about 30%, by weight of the composition.

Any oily components can be used herein. The oily component can meet the conditions described below under the title “PHYSICAL PARAMETERS OF THE PHASES”. When two or more oily components are used as a mixture in the oil phases, the mixture can meet such conditions. Solid oily components can be used as a mixture with liquid oily components, as long as it is fluidized as an oil phase when shaking. In some examples, liquid oily components can be used, and such liquid oily components can have a melting point of 15° C. or less, alternatively 10° C. or less, alternatively 0° C. or less.

Representative oily components useful herein include, for example, Isohexadecane (Density of about 0.79 g/ml), Isopropyl Palmitate (Density of about 0.86 g/ml), Isopropyl Isostearate (Density of about 0.86 g/ml), Caprylic/Capric Triglyceride (Density of about 0.91 g/ml), Isopropyl Lauroyl Sarcosinate (Density of about 0.94 g/ml),Triethylhexanoin (Density of about 0.96 g/ml), Dimethicone 50 cst (Density of about 0.97 g/ml).

### Incompatible Ingredients

In one embodiment, the oil discontinuous phase may contain ingredients which are incompatible to at least one ingredient contained in the aqueous continuous phase.

Combinations of such incompatible ingredients are, for example: Niacinamide and Vitamin A or its derivatives (e.g. Retinyl Propionate); Niacinamide and pH variation (<4 or >7); water and Vitamin C or its derivatives.

For example, by the incorporation of niacinamide in oil phases when Retinyl Propionate is contained in the aqueous continuous phase, the cosmetic composition can reduce the interaction between niacinamide and Retinyl Propionate, and may slow down undesirable chemical reaction of at least one of these ingredients.

### High Oil Absorbance Solid Particulate

Preferably, the dispersed oil discontinuous phase is substantially free of solid particulate having an oil absorbance of 300 ml/100g or more, alternatively oil absorbance of 250 ml/100g or more. “The oil phases being substantially free of high oil absorbance solid particulates” can mean that: the oil phases are free of high oil absorbance solid particulates; or, if the oil phases contain high oil absorbance solid particulates, the level of such high oil absorbance solid particulates is very low. The composition can have a total level of such high oil absorbance solid particulates, if included, of 5% or less, alternatively 3% or less, alternatively 1% or less, alternatively 0.5% or less. In some examples, the total level of such high oil absorbance solid particulates can be 0% by weight of the oil phases.

### Low Oil Absorbance Solid Particulate

The oil phases can contain solid particulate with low oil absorbance, i.e., the oil absorbance of up to about 250 ml/100g, alternatively up to about 200 ml/100g or less, alternatively up to about 150 ml/100g or less.

Such low oil absorbance solid particulates include, for example: some silicone elastomers such as that with tradename KSP-100; some silica for example, that with tradename Godd Ball G-6C (oil absorbance of 30 ml/100g) and that with tradename Godd Ball D-25C (oil absorbance of 90 ml/100g) Mica; TiO₂; Iron oxide;

Any % as long as the oil phase has the viscosity.

### Other Ingredients in Oil Phases

The oil phases may include one or more other ingredients known for use in topical cosmetic compositions, provided that such other ingredients do not unacceptably alter the desired benefits and/or properties of the oil phases. Such other ingredients include, for example those described under the title of “OTHER INGREDIENTS”.

Any ingredients can be incorporated regardless of hydrophobicity/hydrophilicity, as long as such ingredients do not prevent/disturb formation and dispersion of oil discontinuous phase. Any water-soluble, partially water-soluble ingredients (such as those having a water-solubility of about 0.5 g/L or more at 25° C.), water-insoluble chemicals can be added. Preferably, the water-soluble, partially water-soluble ingredients (such as those having a water-solubility of about 0.5 g/L or more at 25° C.), if included, are solid at room temperature.

Representative other ingredients in the oil phases include, for example: Water/oil insoluble particulates such as mica, titanium dioxide, silicone elastomer powder; Water soluble solid materials such as niacinamide, vitamin C, vitamin C derivatives. When the water soluble solid materials are incorporated, the water soluble solid materials may be diffused gradually to the aqueous continuous phase.

Color and/or Reflactive Index

The composition can have a color and/or refractive index of the oil discontinuous phase is different from a color and/or refractive of the aqueous phase, in view of providing aesthetic benefit. The difference in refractive index between the oil phases and the aqueous phase can be 0.03 or more.

### Physical Parameters of the Phases

The aqueous phase and oil phases can meet the physical parameters shown below in Table 1. Details of each parameter are described below.

TABLE 1

| | Aqueous phase containing | Aqueous phase containing | Aqueous phase containing |
|---|---|---|---|
| | hydrophilic thickener w/o | hydrophilic thickener w/ | both of the hydrophilic |
| | associating ability to oil | associating ability to oil | thickeners in left |
| Light density oil | Having G′ max. >5 and | Having G′ max. >1 | Having G′ max. >2.5 |
| phases, i.e., when | having a Cross Point (CP) | | |
| density difference | | | |
| between Aqueous | | | |
| phase and Oil phases | | | |
| is above 0.18 | | | |
| (excluding 0.18) to 0.40 | | | |
| Other oil phases, i.e., | Having G′ max. >4, | Having G′ max. >0, | Having G′ max. >0, |
| when density | alternatively G′ | alternatively G′ | alternatively G′ |
| difference between | max. >5, and having | max. >1 | max. >2.5 |
| Aqueous phase and | a Cross Point (CP) | | |
| Oil phases is from | | | |
| 0 to 0.18 | | | |

Table 1 shows that:
(1) When density difference between the aqueous phase and the oil phases is above 0.18 (excluding 0.18) to 0.40, the aqueous phase meet one of the following conditions:
   (1-1) when the aqueous phase contains a hyrophilic thickener w/o associating ability to oil, the aqueous phase preferably has G′ max. >5 and a Cross Point (CP);
   (1-2) when the aqueous phase contains a hydrophilic thickener w/associating ability to oil, the aqueous phase preferably has G′ max. >1;
   (1-3) when the aqueous phase contains both of: a hydrophilic thickeners w/o associating ability to oil; and a hydrophilic thickeners w/associating ability to oil, the aqueous phase preferably has G′ max. >2.5;
(2) When density difference between the aqueous phase and the oil phases is from 0 to 0.18, the aqueous phase meet one of the following conditions:
   (2-1) when the aqueous phase contains a hydrophilic thickener w/o associating ability to oil, the aqueous phase preferably has G′ max. >4, more preferably G′ max. >5, and preferably has a Cross Point (CP);
   (2-2) when the aqueous phase contains a hydrophilic thickener w/associating ability to oil, the aqueous phase preferably has G′ max. >0, more preferably G′ max. >1;
   (2-3) when the aqueous phase contains both of: a hydrophilic thickeners w/o associating ability to oil; and a hydrophilic thickeners w/associating ability to oil, the aqueous phase preferably has G′ max. >0, more preferably G′ max. >2.5.

### Elasticity Values G′ Max. and G″

The aqueous phase can have a certain “G′ max.” value, depending on the type of hydrophilic thickener contained in the aqueous phase, and also depending on the density difference between the aqueous phase and the oil phases. The details of such “G′ max.” values are shown in Table 1, in view of retain the oil dispersion for longer time.

The elasticity values G′ max. and G″ are measured by using the exact composition of the aqueous phase containing all ingredients used in the aqueous phase. Measurement method and conditions are as follows, and the same method and conditions are applied for the measurement of Cross Point (CP) described below:
Method: Oscillation Stress Sweep Method
Instrument: Discovery HR-2 Rheometer (TA Instrument)
Geometry: 40 mm 2.004° cone plate, Peltier plate Aluminium
Geometry Gap setting: Trim gap offset is 50 μm, Truncation gap is 48 μm
Conditioning-Sample (Step 1)
Temperature set: 25° C.
Oscillation-Amplitude (Step 2)
Temperature Set: 25° C.
Test Parameter: Frequency 1Hz, Logarithmic sweep, Stress 1.0 up to 100 Pa, Points per decade are at least 10.

### Cross Point (CP)

Cross point is where G′ and G″ cross. As shown in Table 1, when the aqueous phase contains hydrophilic thickener thickener w/o associating ability to oil, and especially when the aqueous phase is free of hydrophilic thicker w/associating ability to oil, it is preferred that the aqueous phase has such cross point, in view of retaining the oil dispersion for longer time.

### Density Difference

The oil phases and the aqueous phase can have a certain difference in their densities as shown in Table 1, in view of making the oil discontinuous phase dispersed for longer time.

The density of the oil phases is measured by using the exact composition of the oil phases containing all ingredients used in the oil phases, but excluding solid particulates if used in the oil phases.

The density of the aqueous phase is measured by using the exact composition of the aqueous phase containing all ingredients used in the aqueous phase, but excluding those dispersed and those which do not form continuous phase with an aqueous carrier if such ingredients are used in the aqueous phase.

The densities are measured at 20-25° C.

### Viscosity of the Oil Phases

In some examples, the dispersed oil discontinuous phase can have the following viscosities:

When the oil phases are newtonian, from about 1 cps to about 20,000 cps, alternatively from about 1 cps to about 10,000 cps; and

When the oil phases are non-newtonian, from about 1 cps to about 300,000 cps, alternatively from about 1 cps to about 200,000 cps., alternatively from about 2 cps to about 100,000 cps.

The viscosities of the dispersed oil discontinuous phase are measured by using the exact composition of these oil phases containing all ingredients used in these oil phases.

Measurement method and conditions are as follows:

At room temperature, Brookfield RV Viscometer, T bar, (T-A bar for low viscosity, T-E for high viscosity), spindle speed=5 rpm for both newtonian and non-newtonian oil phases.

### Viscosity of the Aqueous Phase

The aqueous phase can have a viscosity of from about 100 cps to about 50000 cps, alternatively from about 100 cps to about 30000 cps.

The viscosity of the aqueous phase is measured by using the exact composition of the aqueous phase containing all ingredients used in the aqueous phase.

Measurement method and conditions are as follows:

At room temperature, Brookfield RV Viscometer, T bar, (T-A bar for low viscosity, T-E for high viscosity), spindle speed=5 rpm

### Other Ingredients

### Skin Conditioning Agent

Optionally, the composition can further comprise a skin conditioning agent. These agents may be selected from humectants and emollients. The amount of skin-condition agent may range from about 1% to about 50%, alternatively from about 2% to about 40%, alternatively from about 5% to about 30%, by weight of the composition.

Humectants are polyhydric alcohols intended for moisturizing, reducing scaling and stimulating removal of built-up scale from the skin. Typical polyhydric alcohols include polyalkylene glycols and alternatively alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. In some examples, the humectant can be glycerin. When the conditioning agent is an emollient it may be selected from hydrocarbons, fatty acids, fatty alcohols and esters.

### Fatty Alcohol

The compositions herein may include a fatty alcohol. Fatty alcohols refer to high-molecular-weight , straight-chain primary alcohols that have the general structure:

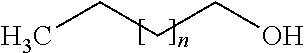

where n=8 to 32.

Fatty alcohols may be natural or synthetic, saturated or unsaturated, branched or straight-chain. Some nonlimiting examples of fatty alcohols commonly used in cosmetic compositions include caprylic, capryl, lauryl, myristyl, cetyl, stearyl, and behenyl alcohols. The fatty alcohols herein may be referred to generically by the number of carbon atoms in the molecule. For example, a “C12 alcohol” refers to an alcohol that has 12 carbon atoms in its chain (i.e., dodecanol).

The fatty alcohol may be included in the compositions herein at 0.0001% to 15% (e.g., 0.0002% to 10%, 0.001% to 15%, 0.025% to 10%, 0.05% to 7%, 0.05% to 5%, or even 0.1% to 3%) by weight of the composition.

### Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B 3 compounds, azelaic acid, butyl hydroxy anisole, gallic acid and its derivatives, hydroquinoine, kojic acid, arbutin, mulberry extract, tetrahydrocurcumin, and mixtures thereof. Use of combinations of whitening agents is also believed to be advantageous in that they may provide whitening benefit through different mechanisms.

When used, the compositions can contain from about 0.1% to about 10%, alternatively from about 0.2% to about 5%, by weight of the composition, of a whitening agent.

Ascorbic acid compounds are useful whitening agents and ascorbyl glucoside can be a preferred derivative of the ascorbic acid compounds.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Particularly suitable examples of conditioning agents include non-volatile silicone fluids other than those described above under the title “SILICONE OIL”, such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization.

### Emulsifiers

When the composition is in the form of an emulsion, it may contain an emulsifier. Emulsifiers may be nonionic, anionic, cationic, or zwitterionic. Some non-limiting examples of emulsifiers are disclosed in U.S. Pat. Nos. 3,755,560, 4,421,769, U.S. Publication No. 2006/0275237 and McCutcheon's Detergents and Emulsifiers, North American Edition, pages 317-324 (1986). Suitable examples of emulsifiers include non-ionic stearic acid derivative emulsifiers such as Polysorbate-20, glyceryl stearate, sorbitan stearate, PEG-100 stearate, Steareth-2, Steareth-21, glycereth-25 pyrrolidonecarboxylic acid isostearate and combinations of these.

Alternatively, the composition can be substantially free of emulsifiers, alternatively the composition can be free of emulsifiers, i.e., contains 0% of emulsifiers.

### Other Optional Ingredients.

Compositions herein may include one or more optional ingredients known for use in topical cosmetic compositions, provided that the optional components do not unacceptably alter the desired benefits of the composition. In some instances, it may be desirable to select cosmetic actives that function via different biological pathways so that the actives do not interfere with one another. When the composition is in the form of an emulsion, the additional ingredients should not introduce instability into the emulsion (e.g., syneresis). For example, it may be desirable to select optional ingredients that do not form complexes with other ingredients in the composition, especially pH sensitive ingredients like vitamin B3 compounds, salicylates and peptides.

The additional ingredients should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some nonlimiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Some particularly suitable examples of additional ingredient include one or more skin care actives selected from the group consisting of vitamin B3 compounds (e.g., niacinamide), vitamin B5 compounds (e.g., d-panthenol), n-acyl amino acids (e.g., undecylenoyl phenylalanine), vitamin E compounds (e.g., tocopheryl acetate), palmitoylated dipeptides (e.g., palmitoyl-lysine-threonine), palmitoylated pentapeptides (e.g., palmitoyl-lysine-threonine-threonine-lysine-serine), vitamin A compounds (e.g., retinol and retinyl propionate), and combinations thereof. Other non-limiting examples of optional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/0092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Sucrose esters may be used herein. Such sucrose ester can be a blend of two or more sucrose esters, wherein the two or more sucrose esters are present at a ratio of any one sucrose ester to another of 1:10 to 1:1 (e.g., 1:7, 1:5, 1:3, or 1:2). In some instances, the sucrose ester may be a blend of sucrose laurate and sucrose dilaurate, wherein sucrose laurate is present at 50% to 80%, by weight of the sucrose ester, and the sucrose dilaurate is present at 20% to 45%, by weight of the sucrose ester. Alternatively, the sucrose ester may be a blend of sucrose laurate, sucrose dilaurate and sucrose trilaurate, wherein the sucrose dilaurate is present at 35% or more, by weight of the sucrose ester. A particularly suitable example of a sucrose ester for use herein is Sucrose Dilaurate BC10034 available from BASF.

### Method of Use of the Cosmetic Composition

The method of use herein includes identifying a target portion of skin on a person in need of treatment and applying the composition to the target portion of skin over the course of a treatment period. The target portion of skin may be on a facial skin surface such as the forehead, perioral, chin, periorbital, nose, and/or cheek) or another part of the body (e.g., hands, arms, legs, back, chest). The person in need of treatment is one whose skin exhibits signs of oxidative stress, such as fine lines, wrinkles, hyperpigmentation, uneven skin tone, and/or other visible skin features typically associated with aging. In some instances, the target portion of skin may not exhibit a visible sign of skin aging, but a user (e.g., a relatively young user) may still wish to target such an area of skin, if it is one that typically develops such issues as a person age. In this way, the present method may be used as a preventative measure to delay the onset of visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or line) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied broadly to one or more skin surfaces. In certain embodiments, the compositions herein may be used as part of a multi-step beauty regimen, wherein the present composition may be applied before and/or after one or more other compositions.

### Combinations

A cosmetic composition comprising an aqueous continuous phase and an oil discontinuous phase dispersed in the aqueous continuous phase, wherein the oil discontinuous phase has a particle size of from about 0.01 mm to about 20 mm, and wherein the oil discontinuous phase is dispersed for at least 24 hours, wherein the composition is free of polymeric encapsulation.

The cosmetic composition of the preceding feature, wherein the oil discontinuous phase has a particle size of preferably from about 0.05 mm to about 10 mm, alternatively from about 0.1 mm to 5 mm,

The cosmetic composition of any of the preceding features, wherein the oil discontinuous phase is dispersed preferably for at least 3 days, alternatively for at least for 5 days, alternatively for at least 1 week.

The cosmetic composition of any of the preceding features, wherein a color and/or refractive index of the oil phases is different from a color and/or refractive of the aqueous phase.

The cosmetic composition of any of the preceding features, wherein the aqueous phase and the oil discontinuous phase meet the following conditions:
(1) When density difference between the aqueous phase and the oil phases is above 0.18 (excluding 0.18) to 0.40, the aqueous phase meet one of the following conditions:
   (1-1) And when the aqueous phase contains a hydrophilic thickener w/o associating ability to oil, the aqueous phase has G′ max. >5 and a Cross Point (CP);
   (1-2) And when the aqueous phase contains a hydrophilic thickener w/associating ability to oil, the aqueous phase has G′ max. >1;
   (1-3) And when the aqueous phase contains both of: a hydrophilic thickeners w/o associating ability to oil; and a hydrophilic thickeners w/associating ability to oil, the aqueous phase has G′ max. >2.5;
(2) When density difference between the aqueous phase and the oil phases is from 0 to 0.18, the aqueous phase meet one of the following conditions:
   (2-1) And when the aqueous phase contains a hydrophilic thickener w/o associating ability to oil, the aqueous phase has G′ max. >4, and has a Cross Point (CP);
   (2-2) And when the aqueous phase contains a hydrophilic thickener w/associating ability to oil, the aqueous phase has G′ max. >0;
   (2-3) And when the aqueous phase contains both of: a hydrophilic thickeners w/o associating ability to oil; and a hydrophilic thickeners w/associating ability to oil, the aqueous phase has G′ max. >0.

EXAMPLES
Cosmetic composition (percent by weight)

| | | Ex. 1 | | Ex. 2 | | CEx. i | | Ex. 3 | | Ex. 4 | | Ex. 5 | |
|---|---|---|---|---|---|---|---|---|---|---|---|---|---|
| Oil | Isopropyl | 10 | | | | | | | | | | | |
| phase | Isostearate | | | | | | | | | | | | |
| ingredients | Dimethicone | | | 10 | | | | 10 | | 0.7 | | | |
| | 50 cst | | | | | | | | | | | | |
| | Dimethicone | | | | | | | | | 3.5 | | | |
| | 5 cst | | | | | | | | | | | | |
| | Dow | | | | | | | | | 0.5 | | | |
| | corning | | | | | | | | | | | | |
| | 1503 *1 | | | | | | | | | | | | |
| | KF56A *2 | | | | | | | | | 0.8 | | | |
| | Retinyl | | | | | | | | | 0.3 | | | |
| | Propionate | | | | | | | | | | | | |
| | Beta | | | | | | | | | 0.00050 | | | |
| | Carotene | | | | | | | | | | | | |
| | Caprylic/Capric | | | | | | | | | | | 3 | |
| | Triglyceride | | | | | | | | | | | | |
| | Isohexadecane | | | | | 10 | | | | | | | |
| | Niacinamide | | | | | | | | | | | 2 | |
| | Dextrin | | | | | | | | | | | 0.06 | |
| | Palmitate | | | | | | | | | | | | |
| Oil | Density | 0.19 | | 0.09 | | 0.26 | | 0.09 | | 0.07 | | 0.08 | |
| phase | difference | | | | | | | | | | | | |
| proper | from | | | | | | | | | | | | |
| ties | Aqueous | | | | | | | | | | | | |
| | phase | | | | | | | | | | | | |
| | Viscosity | 1-5 | cps | 50 | cps | 280 | cps | 50 | cps | 160 | cps | 69000 | cps |
| Aqueous | Glycerin | 2.7 | | 2.7 | | 2.7 | | 2.7 | | 3 | | 1.5 | |
| phase | Xylitol | 4.23 | | 4.23 | | 4.23 | | 4.23 | | | | | |
| ingredients | Niacinamide | 4.5 | | 4.5 | | 4.5 | | 4.5 | | 5 | | | |
| | Phenoxyethand | 0.225 | | 0.225 | | 0.225 | | 0.225 | | 0.15 | | | |
| | Butylene | | | | | | | | | | | 4 | |
| | Glycol | | | | | | | | | | | | |
| | D-panthenol | | | | | | | | | | | 0.5 | |
| | Sodium | | | | | | | | | | | 0.05 | |
| | Benzoate | | | | | | | | | | | | |
| | Disodium | | | | | | | | | 0.05 | | | |
| | EDTA | | | | | | | | | | | | |
| | Symsave-H *3 | | | | | | | | | 0.15 | | | |
| | pH | | | | | | | | | | | 6.27 | |
| | modifiers | | | | | | | | | | | | |
| | Sepimax | | | | | | | | | 0.45 | | 0.75 | |
| | Zen *4 | | | | | | | | | | | | |
| | Ultrez 20 | 0.045 | | | | | | | | | | | |
| | *5 | | | | | | | | | | | | |
| | Pemulen | | | 0.045 | | 0.045 | | 0.027 | | 0.15 | | | |
| | TR-2 *6 | | | | | | | | | | | | |
| | Ultrez 10 | | | | | | | 0.063 | | | | | |
| | *7 | | | | | | | | | | | | |
| | Aminomethyl | 0.0135 | | 0.0135 | | 0.0135 | | 0.0157 | | 0.045 | | | |
| | Propanol | | | | | | | | | | | | |
| | Deionized | q.s. to | | q.s. to | | q.s. to | | q.s. to | | q.s. to | | q.s. to | |
| | water | 100% | | 100% | | 100% | | 100% | | 100% | | 100% | |
| Aqueous | G′ max | 18.0 | Pa | 0.6 | Pa | 0.6 | Pa | 14.8 | Pa | 17.9 | Pa | 1.6 | Pa |
| phase | CP | 2.5 | Pa | None | | None | | 2.9 | Pa | 11 | Pa | 4.2 | Pa |
| properties | Viscosity | 6533 | cps | 567 | cps | 567 | cps | 5513 | cps | 5600 | cps | 2080 | cps |

| | | | | | | | | | | | | |
|---|---|---|---|---|---|---|---|---|---|---|---|---|
| Particle size | 150 | μm | 290 | μm | 160 | μm | 230 | μm | 80 | μm | 160 | μm |
| (volume-based) *i) | | | | | | | | | | | | |
| of oil discontinuous | | | | | | | | | | | | |
| phase | | | | | | | | | | | | |

| | | | | | | |
|---|---|---|---|---|---|---|
| Duration of the | More than | More than | Fail in | More than | More than 1 | More than |
| sustained | 1 month | 1 month | 24 hours | 1 month | month | 1 month |
| dispersion of oil | | | | | | |
| discontinuous | | | | | | |
| phase *ii) | | | | | | |

Measurements
*i) Particle Size Measurement
Method - Laser Scattering Particle Size Distribution Analysis
Equipment - HORIBA LA-950j
Sample Preparation - Fraction Cell
*ii) Measured by visual observation of the composition
Ingredients
*1 Dow corning 1503 INCI: Dimethicone & Dimethiconol
*2 KF-56A INCI: Diphenylsiloxy Phenyl Trimethicone
*3 Symsave-H INCI: Hydroxyacetophenone
*4 Sepimax Zen INCI: Polyacrylate crosspolymer-6
*5 Ultrez 20 INCI: Acrylates/C10-30 Alkyl Acrylate Crosspolymer
*6 Pemulen TR-2 INCI: Acrylates/C10-30 Alkyl Acrylate Crosspolymer
*7 Ultrez 10 INCI: Carbomer

Ex. 1 through Ex. 5 are skin care cosmetic compositions as described herein. CEx. i is a skin care cosmetic composition provided as a comparative example. As shown in the above tables, the compositions described herein can provide prolonged dispersion of the dispersed oil phases.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising an aqueous continuous phase and an oil discontinuous phase dispersed in the aqueous continuous phase, wherein the oil discontinuous phase comprises a particle size of from about 0.01 mm to about 20 mm, wherein the oil discontinuous phase is dispersed for at least 24 hours, and wherein the composition is free of polymeric encapsulation.

2. The cosmetic composition of claim 1, wherein the oil discontinuous phase has a particle size of from about 0.05 mm to about 10 mm

3. The cosmetic composition of claim 2, wherein the oil discontinuous phase has a particle size of from about 0.1 mm to 5 mm

4. The cosmetic composition of claim 1, wherein the oil discontinuous phase is dispersed for at least 3 days.

5. The cosmetic composition of claim 4, wherein the oil discontinuous phase is dispersed for at least 5 days,

6. The cosmetic composition of claim 5, wherein the oil discontinuous phase is dispersed for at least 1 week.

7. The cosmetic composition of claim 1, wherein a color and/or refractive index of the oil phases is different from a color and/or refractive of the aqueous phase.

8. The method of claim 1, wherein the aqueous phase comprises a hydrophilic thickener without associating ability to oil; wherein the density difference between the aqueous phase and the oil phase is from 0 to 0.4; and wherein the aqueous phase comprises a G′ max. of greater than 4 and a Cross Point.

9. The method of claim 8, wherein the density difference between the aqueous phase and the oil phase is greater than 0.18 and less than or equal to 0.4; and wherein the aqueous phase has a G′ max. greater than 5.

10. The method of claim 1, wherein the aqueous phase comprises a hydrophilic thickener with associating ability to oil; wherein the density difference between the aqueous phase and the oil phase is from 0 to 0.4; and wherein the aqueous phase comprises a G′ max. of greater than 0.

11. The method of claim 10, wherein the density difference between the aqueous phase and the oil phase is greater than 0.18 and less than or equal to 0.4; and wherein the aqueous phase has a G′ max. greater than 1.

12. The method of claim 1, wherein the aqueous phase comprises a hydrophilic thickener with associating ability to oil and a hydrophilic thickener without associating ability to oil; wherein the density difference between the aqueous phase and the oil phase is from 0 to 0.4; and wherein the aqueous phase comprises a G′ max. of greater than 0.

13. The method of claim 12, wherein the density difference between the aqueous phase and the oil phase is greater than 0.18 and less than or equal to 0.4; and wherein the aqueous phase has a G′ max. greater than 2.5.
